(19) United States
(12) Plant Patent Application Publication
(10) Pub. No.: US 2003/0033654 P1
(43) Pub. Date: Feb. 13, 2003

(54) Petunia plant named 'keidopuel'

(76) Inventors: Hiroshi Hirabayashi, Yachiyo-shi (JP); Eriko Hirabayashi, Yachiyo-shi (JP); Minako Hirabayashi, Yachiyo-shi (JP); Kanako Hirabayashi, Yachiyo-shi (JP)

Correspondence Address: KLARQUIST SPARKMAN, LLP, 121 SW SALMON STREET, SUITE 1600, PORTLAND, OR, 97204, US

(21) Appl. No.: 09/929,155

(22) Filed: Aug. 13, 2001

(51) Int. Cl. A01H 5/00

(52) U.S. Cl. PLT/356

(57) ABSTRACT

Petunia plant having a vivid purple coloration.

The present invention relates to a new and distinct variety of petunia which was originated by me by crossing two unnamed seedlings, each of whose parents were also unnamed seedlings, none of which are patented.

### BACKGROUND OF THE INVENTION

The primary objective of this breeding was to produce a new petunia variety having large flowers of a vivid purple color and a plant having a strong growth habit. The objective was substantially achieved

### DETAILED DESCRIPTION OF THE INVENTION

Asexual reproduction of this new variety by cuttings, as performed at Yachiyo Farms, Keisei Rose Nurseries, Inc., 755 Owaciashinden, Yachigo-shi, Chiba-Ken, Japan and at Somis, Calif., shows that the foregoing and all other characteristics and distinctions come true to form and are established and transmitted through succeeding propagations.

The accompanying illustrations show a typical specimen of this new variety, depicted in color as nearly true as it is reasonably possible to make the same in a color illustration of this character:

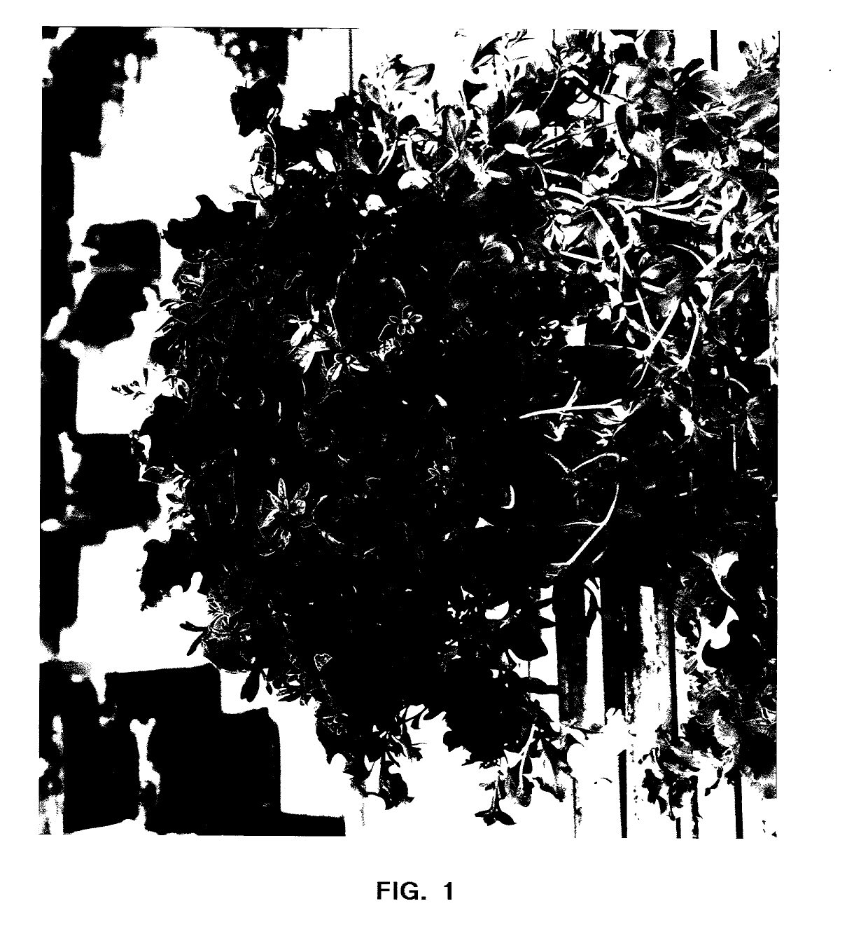

FIG. 1 is a photograph of a mature plant; and

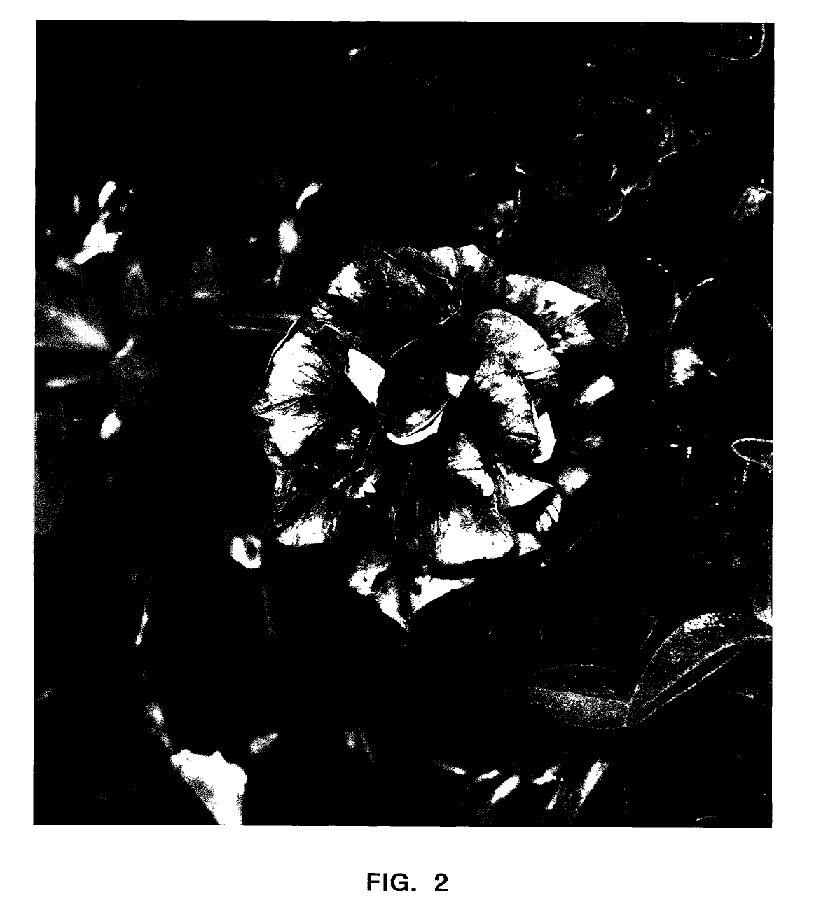

FIG. 2 is an enlarge view of a single blossom as it appears on the plant.

The following is a detailed description of my new rose cultivar with color descriptions using terminology in accordance with The Royal Horticultural Society (London) colour chart, except where ordinary dictionary significance of color is indicated.

Parentage:

Seed parent.—Unnamed seedling (unpatented).

Pollen parent.—Unnamed seedling (unpatented).

Classification:

Botanical.—Petunia hybrida.

Commercial.—Petunia.

Observations made from specimens grown in a garden environment at Somis, Calif. from July, 2000 to December, 2000.

### PLANT

Growth.—Spreading.

Growth habit.—Vigorous, free branching.

Mature plant.—Height: 10-15 cm. Spreading area: 30-40 cm in diameter.

Blooming period.—April to October.

Stem.—Overall Length: 11-20 cm. Main Stem: about 20 cm. Laterals: 11-15 cm. Thickness: 2.0-2.5 mm. Color: Yellow-Green Group 146B. Pubescence: Medium Mode of branching: Abundant Length of Internode: 3-4.5 cm.

Leaf.—Arrangement on stem: Slightly upward. Shape: Elliptic. Size: 3.5-5.0 cm. Thickness: About 1 mm. Color: Upper Surface: Yellow-Green Group 146A Lower Surface: Yellow-Green Group 146A Phyllotaxis: Perpendicular Pubescence: Medium.

Flower:

Size.—2 ¾-3 ¼ inches in diameter.

Type.—Double.

Shape.—Medium corolla tube. Tip obverse.

Color.—The upper sides of the petals are vivid Red-Purple Group 74A. The reverse sides of the petals are Purple Group 76C at the time of petal opening but changing as the petal ages progressively to Purple Group 76B, Purple Group 75C, Red-Purple Group 73C and finally to Red-Purple Group 68D before petal shrivels.

Peduncle.—Length: 1.5-2.5 cm. Color: Yellow-Green Group 146C.

Calyx.—Number of sepals: 5 Length: 11-13 mm. Color: Yellow-Green Group 146C.

Reproductive Parts:

Anthers.—Color: Blue-Green Group 116B.

Filaments.—Color: Yellow-Green Group 150D.

Pistil.—Quantity: One. Color: Greyed-Yellow Group 160D.

Stamens.—Quantity: 3-5.

Disease Resistance: Resistant to mildew under normal growing conditions.

Response to Climatic conditions: Hardy to Zone 10.

I claim:

1. A new and distinct variety of petunia, substantially as herein shown and described, characterized particularly by its vivid purple coloration.
